(19) United States
(12) Patent Application Publication
(10) Pub. No.: US 2021/0236177 A1
(43) Pub. Date: Aug. 5, 2021

(54) SURGICAL DEVICE OF REPERE

(71) Applicant: MT ORTHO S.R.L., Aci Sant'Antonio CT (IT)

(72) Inventor: Giovanni Federico NICOLETTI, Aci Castello CT (IT)

(73) Assignees: Giovanni Federico NICOLETTI, Aci Castello CT (IT); MT ORTHO S.R.L., Aci Sant'Antonio CT (IT)

(21) Appl. No.: 17/052,708

(22) PCT Filed: May 8, 2019
(86) PCT No.: PCT/IB2019/053764

§ 371 Date: Nov. 3, 2020

(30) Foreign Application Priority Data
May 10, 2018 (IT) 102018000005244

(51) Int. Cl. A61B 17/70; A61B 34/20

(52) U.S. Cl. CPC A61B 17/7067; A61B 2034/2068; A61B 34/20; A61B 17/7058

(57) ABSTRACT

The present invention relates to a surgical device of repère configured to be connected to a vertebral spinous process of a patient. The main body includes a skin contact element, having an abutment surface shaped to abut on the skin overlying the vertebral spinous process; handling means connected to the skin contact element, configured to allow the adjustment of the coupling between the device and the vertebral spinous process; and coupling means with the spinous process of the patient, connected to the skin contact element at the abutment surface and shaped to penetrate the external surface of the vertebral spinous process and insert at least partially inside thereof. The surgical device further has position indicating means connected or connectable to the main body to indicate the penetration point of the-coupling means in the vertebral spinous process.

### TECHNICAL FIELD OF THE INVENTION

The present invention relates to a surgical device of repère, apt to provide a visual spatial and radiologically identifiable reference of the position of a vertebral spinous process.

The device the present invention relates to is meant in particular as help to a surgical operator in performing operations of the spinal column. In particular, the device can be advantageously used during the carrying-out of operations of arthrodesis performed with the help of intraoperative tomographs (for example O-ARM).

### BACKGROUND

Thanks to the technological development which has occurred during the most recent years, the spinal surgery can avail of the intraoperative use of computerized devices (tomographs) configured to provide 3D images of the treated spinal districts.

In particular, such tools help the surgeon during operations which provide the implantation of screws of vertebral stabilization systems. Such screws have to be implanted according to penetration trajectories and positions predetermined by the surgeon itself.

The correct positioning of the screws is a crucial factor for the success of the operation, as should these be placed according to trajectories different from the predetermined ones, this would involve the risk of serious lesions of the column nervous structures, as well as lesions of vital veins and arteries and of the same bone structures.

The computerized devices known in the state of art allow to some extent to monitor the positioning of such screws to verify that it is correct, that is it meets the criteria predetermined by the surgical operator.

To this purpose, the above-mentioned devices generally are interfaced to neuro-navigation systems (for example of the O-ARM/Navigator type) which allow the surgeon to follow the route of the screws of the stabilization systems inserted into the vertebrae in the three space sizes.

By means of the help of such computerized control apparatuses the risk that the screws arrange according to not correct routes is reduced to some extent.

However, errors in detecting the trajectories of the above-mentioned screws performed by means of the devices of known type are possible. Such possibility is inherent in the fact that the above-mentioned computerized devices are calibrated with respect to reference systems (‘reference frame’) which are positioned at the beginning of the operation. The positioning of such reference systems affects strongly the accuracy of the detection of the screws' position.

It is recognized that the above-mentioned reference devices can be subjected to dislocations during the operation, which, even if minimum, are sufficient to induce the surgeon in very serious errors.

The only way which can be used today to avoid such errors is to perform, during the operation, several checks of the calibration of the computerized devices which provide the 3D images of the spinal districts under surgical treatment, with periodical radiological acquisition of images during the operation.

This inevitably involves an increase in surgical time, affecting both the surgeon and the patient.

Moreover, such repeated calibration checks involve for the patients and for the whole involved medical personnel a dangerous overexposure to radiations.

US20110218546A1 describes a surgical device comprising a fixed portion intended to be connected to a bone of a patient, a mobile portion with respect to said fixed portion and position indicating means, in the form of a tracing element apt to be detected by a navigation system.

US20170348061A1 describes a surgical robot associated to reference means which can be applied to the body district of a patient, comprising an array having four markers apt to assist the spatial coordination of the robot itself with respect to the patient.

### SUMMARY OF THE INVENTION

The technical problem placed and solved by the present invention is then to provide a surgical device allowing to obviate the drawbacks mentioned above with reference to the known art.

The above-mentioned drawbacks are solved by a surgical device of repère according to claim 1.

Preferred features of the present invention are set forth in the depending claims.

The present invention provides a device suitable to provide a fixed visual reference point which can be identified by the tomographs, helping the surgeon to perform operations of the spinal column.

To this purpose, the device comprises a skin contact element configured for abutting on the skin of the patient, at the spinous process of a vertebra, and coupling means configured to penetrate and insert within the above-mentioned spinous process.

The coupling means is configured to implement a connection with the spinous process which is apt to remain fixed during the whole duration of the operation. The coupling means has a main development direction, or longitudinal development direction, which coincides with the preferential direction of insertion of the same in the spinous process.

The skin contact element in turn is connected to handling means of the device itself, and above all to position indicating means.

The position indicating means is configured to indicate the penetration point of the coupling means in the spinous process. Therefore, when the device is in use, the position indicating means is arranged along the main development direction, or longitudinal development direction, of the coupling means.

The device according to the present invention allows to provide the surgeon a point of repère, in other words, a spatial visual reference of a particular anatomic element, which keeps constant during the carrying-out of the operation.

In particular, the use of the device can be provided in addition to the reference systems of traditional type, associated to the computerized devices which provide 3D images of the involved spinal districts.

Advantageously, the device of the invention allows to be able to check, without interruptions, in real time, the correct calibration of neuro-navigation systems during the carrying-out of surgical operations of the spinal column.

Still more advantageously, the continuous monitoring allowed by means of the device does not provide the need for new radiological acquisitions. Therefore, the medical personnel and the patient are not exposed to additional doses of radiations.

According to a particularly preferred embodiment of the invention, the above-mentioned coupling means comprises an oblong engagement element, shaped like a screw, provided with external thread. In particular, such engagement element has a free end provided with a sharp point, which favours the penetration of the coupling means within the vertebral spinous process.

The particular screw-like shape of the engagement element allows to implement a fastening mode of the device for screwing within the spinous process, to implement a threaded coupling (at least regarding the portion of the device of the invention).

Such coupling, even if reversible, is characterized by a high precision in mutual position of the coupled bodies (spinous device and process), which keeps constant in time. In the specific case, the coupling configuration makes it very improbable, if not impossible, even the minimum dislocation of the device with respect to its seat of insertion in the spinous process.

Still, according to particularly advantageous variants of the invention, the device is made partially or wholly of radiopaque material, that is material which does not make the X rays to pass through it.

Advantageously, thanks to the use of the device according to the present invention, the surgeon can control the exact calibration of the tools in few seconds, exclusively by resting the ‘passive planner’ or the ‘pack needle’ on the vertex of the device according to the present invention.

Other advantages, features and use modes of the present invention will result evident from the following detailed description of some embodiments, shown by way of example and not for limitative purposes.

### BRIEF DESCRIPTION OF THE FIGURES

The enclosed Figures will be referred to, wherein:

FIG. 1 shows a front perspective view of a first preferred embodiment of a surgical device according to the present invention;

FIG. 2 shows an enlargement of an upper portion of the device of FIG. 1;

FIGS. 3A to 3C show, respectively in sequence, perspective views of application phases of the device of FIG. 1 to a spinous process, respectively a positioning phase, a penetration phase and a phase for connecting position indicating means to the main body of the device;

FIG. 4 shows an enlarged view, in exploded configuration, of a detail of FIG. 3C, wherein an upper portion of the device of FIG. 1 can be seen, including position indicating means.

The above-mentioned Figures are to be meant exclusively by way of example and not for limitative purposes. In particular, the lengths, thicknesses and mutual proportions between the elements represented therein can have different—larger or smaller—size parameters—with respect to what described in the following section, by way of representation clarity.

### DETAILED DESCRIPTION OF PREFERRED EMBODIMENTS

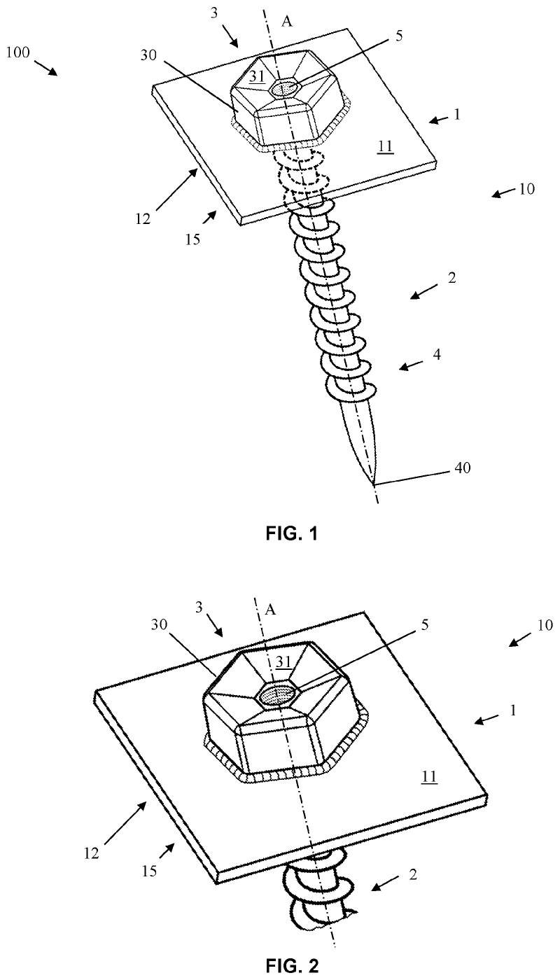

By firstly referring to FIGS. 1 and 2, a preferred embodiment of a surgical device according to the present invention is designated as a whole with 100. In particular, the device of the invention is suitable to provide a visual spatial reference for helping to carry out spinal operations, such as operations of arthrodesis.

The device 100 substantially comprises a main body 10, which, in turn, comprises a skin contact element 1, coupling means 2 with the spinous process of the patient and handling means 3.

The skin contact element 1 is configured to abut on the skin overlying a vertebral spinous process, at least at its own abutment surface 15 suitably configured to the purpose.

To this purpose, according to the preferred embodiment shown in the enclosed Figures, the skin contact element 1 preferably has a plate-like shape, comprising a lower face 12 and an upper face 11, opposite to each other. Preferably, the skin contact element 1 has a quadrangular or square shape in plan. According to the preferred embodiment shown in the enclosed Figures, the device 100 comprises a skin contact element 1 constituted by a square-plan plate wherein each side is about 1.5 cm, whereas the plate thickness is equal to about 3 mm.

The lower face 12 comprises the above-mentioned abutment surface 15, shaped to abut—directly or indirectly, that is after mediation of one or more additional elements—on the skin overlaying the vertebral spinous process of the patient. The abutment surface 15 preferably has a quadrangular or square profile in plan and still more preferably has a substantially flat or planar development.

Moreover, the main body 10 comprises handling means 3 connected to the skin contact element 1, configured to allow the surgeon to be able to adjust the coupling between the device 100 itself and the spinous process. Moreover, the handling means 3 eases the operator in handling the device 100. Such means 3 can include a head element 30, for example shaped like a hexagon nut (as visible in FIG. 2), configured to be able to engage with a tightening wrench or other equivalent means.

Such means 3, with particular reference to the head element 30, preferably is arranged at the upper face 11 of the skin contact element 1, in particular it is opposed to the abutment surface 15, so as to remain external to the spinous process and always to allow the operator to adjust and extract the device 100, during and after the surgical operation.

In particular, the head element 30 is connected to the upper face 11 in a permanent manner, for example by means of welding, to guarantee a firm and resistant connection.

The head element 30 preferably comprises a concave external upper surface 31, in particular the peripheral edges of the external upper surface 31 result to be more projecting—with reference to the skin contact element 1—of the central portion of the same external upper surface 31, and therefore farer from the upper face 11. According to the preferred embodiment shown in the enclosed Figures, the maximum thickness of the head element 30, that is the distance between the peripheral edges of the external upper surface 31 and the upper face 11, is equal to about 5 mm. Such distance is of course reduced at the central portion of the upper face 11 should the latter have a concave configuration, wherein the concavity is maximum indeed at the above-mentioned central portion.

As said before, the main body 10 further comprises coupling means 2 with the spinous process of the patient, which is connected to the skin contact element 1 preferably at the lower face 12, or still more preferably at the abutment surface 15.

The coupling means 2 is shaped to penetrate the external surface of the spinous process and to insert at least partially inside thereof. To this purpose, the coupling means 2 comprises at least a protruding element 4, preferably having an overall oblong shape or stem-like shape, apt to penetrate the spinous process.

The coupling means 2, and in particular at least a protruding element 4, has a main development axis, or longitudinal development axis, which preferably coincides with its own axis of symmetry A, still more preferably an axis of central symmetry A.

Hereinafter, the main development or longitudinal axis of the means 2 or of the protruding element 4 will be referred to as main development or longitudinal direction.

In order to ease the penetration of at least a protruding element 4 within the spinous process, such protruding element 4 preferably is provided with a free terminal end 40, opposed to the abutment surface 15, having a sharp point. Such point, which has no threads, develops along the axis A for a length preferably comprised between about 3 and 8 mm.

With the purpose of increasing the contact area of the coupling means 2 with the spinous process, while maintaining reduced the overall sizes, such at least a protruding element 4 comprises preferably a threaded external surface. According to such embodiment variant, shown in the enclosed Figures, the at least a protruding element 4 is substantially comparable to the stem of a screw and it has in particular a circular cross section, having constant diameter and preferably equal to 2 mm. By way of example, the threaded surface extends along the axis A for a length comprised between about 1 and 12 cm along the above-mentioned main development direction, proceeding from the abutment surface 15 towards the free terminal end.

The length of the sharp point 40 and of the external threaded surface 4, considered along the axis A, are variable compared to the height of the involved spinous process and to the thickness of the adipose layer interposed between apex of the spinous process and cutaneous surface. Such measures can be determined by analysing the X-ray diagnostic images, CAT, Magnetic Resonance or other diagnostic system.

Moreover, preferably, the abutment surface 15 is arranged to surround at least partially the coupling means 2 in order to favour a stable connection with the spinous process and to avoid unwished motions of the device 100 once implanted.

In the particular embodiment variant shown in the enclosed Figures, which provides the presence of a single protruding element 4, the latter has a main development direction according to its own axis of symmetry A, which in particular coincides with an axis of central symmetry of the device 100 considered as a whole, at least according to a configuration wherein the skin contact element 1 and the handling means 3 are centred with respect to such axis A. In other words, the device 100 can be symmetric with respect to the axis A.

In particular, the axis of symmetry A develops along a longitudinal direction, substantially orthogonal with respect to the abutment surface 15. The axis of symmetry A, or generally the main development direction—or longitudinal development direction—of at least a protruding element 4, defines the preferential direction of insertion of the coupling means 2 in the spinous process.

Still, the device 100 comprises position indicating means 60, arranged to designate the penetration point of the coupling means 2 in the spinous process.

The position indicating means 60 is configured to designate the penetration point of the coupling means 2, and in particular of the protruding element 4 in the spinous process. Therefore, when it is connected to the main body 10, the position indicating means 60 is arranged along the main development direction, or longitudinal development direction, of the coupling means 2 or of the protruding element 4, the latter being the preferential direction of insertion of the same in the spinous process.

In the particular embodiment variant shown in the enclosed Figures, providing the presence of a single protruding element 4, the position indicating means 60 is configured to connect to the main body 10 at the axis of symmetry A of such protruding element 4, which coincides with the main development or longitudinal direction of the protruding element 4. Preferably, the means 60 is connected or can be connected to the main body 10 in centred position with respect to the latter, and in each case so as to remain well visible and still external to the back of the patient, even after the connection of the device 100 to the spinous process, to implement a point of repère.

According to the preferred embodiment illustrated in the enclosed Figures, at a central portion of the head element 30, or better at a central portion of the external upper surface 31, at least a seat is obtained, configured to engage with, or receive, the position indicating means 60. Such seat is preferably shaped to implement one hole 5. The functionality assigned to the hole 5 is that of allowing the reversible engagement with respective position indicating means 60.

According to what already described, the hole 5 is implemented at the main development or longitudinal direction of the protruding element 4, which corresponds to the axis of symmetry A shown in the enclosed Figures.

In particular, the position indicating means 60 can include a plug element or cap 6 apt to act as point of repère, configured to engage with the hole 5.

FIG. 4 shows a preferred embodiment of such cap 6. The cap 6 has a head portion 7 and a stem portion 8. Preferably, the head portion 7 has substantially spherical geometry, preferably with diameter equal to about 2 mm.

The stem portion 8 is shaped to engage with the hole 5. In particular, the cap 6 can be coupled with the main body 10 by inserting said stem portion within the hole 5. With the purpose of implementing a reversible but stable coupling between the cap 6 and the main body 10 (in particular between the cap 6 and the head element 30), the hole 5 can have an inner thread and the stem portion 8 can have a respective external thread. The stem portion 8 has a length preferably equal to 5 mm, which coincides with a respective minimum depth of the hole 5.

Once the cap 6 is connected to the main body 10, in turn inserted into a spinous process, the head portion 7 implements for the surgeon a visual reference point—point of repère—helping him/her during the carrying-out of spinal interventions.

Alternatively, modes for connecting at least a cap 6 to the main body 10, different from the above-described one, can be provided, wherein the cap 6 is fastened reversibly to the main body 10 or directly to the skin contact element 1.

With reference to what already described, the external upper surface face 31 of the head element 30 is preferably concave to allow to beat—for example with a surgical hammer—on the above-mentioned external upper surface 31, with the purpose of implementing the penetration of the main body 10 in the spinous process, without the beating involves the hole 5 and runs the risk of jeopardizing the capability of engaging with the cap 6, for example by ruining the thread.

The material the device 100 is made of, preferably is a radiopaque material, still more preferably high-tensile and bio-compatible material, for example comprising at least partially steel and/or titanium.

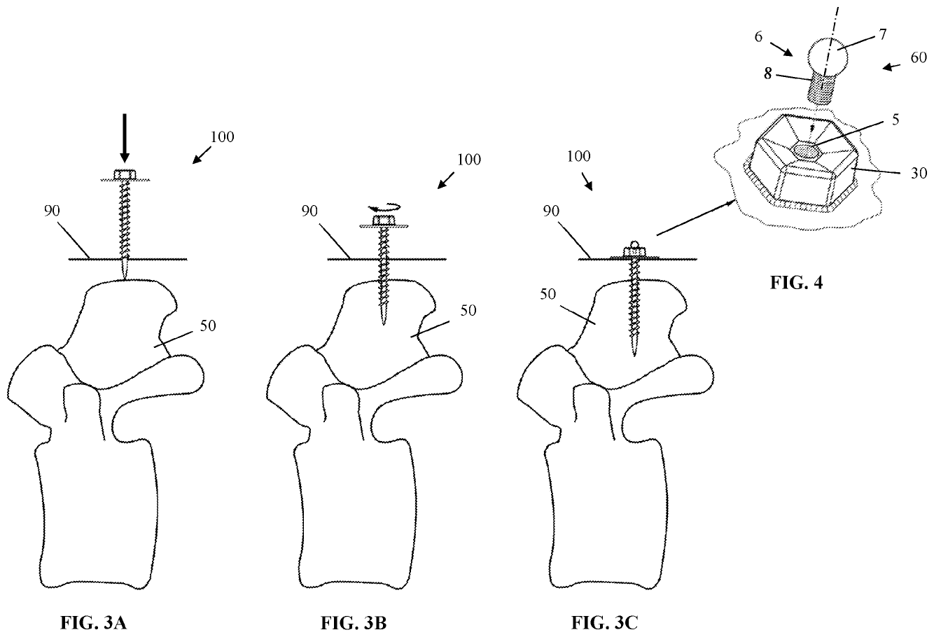

With reference to FIGS. 3A, 3B and 3C, the application phases of a preferred embodiment of the device 100 to the vertebral spinous process 50 of a patient are described hereinafter. The phases are numbered according to the chronological order for performing the same.

The result obtained by applying the device to the patient is to provide to the surgeon a visual indication of a spatial reference point identifiable by the tomograph, which remains fixed for the whole operation duration, in order to monitor the correct calibration of computerized systems, for example of O-ARM/Navigator type.

### Phase 1—Positioning (FIG. 3A)

The already anaesthetized patient is positioned on the operating table. The surgical device 100 according to the present invention is put near the skin 90 at a predetermined spinous process 50 of the patient. Such spinous process 50 is different from the one which will house the traditional reference system.

In particular, the sharp end 40, by perforating the skin 90, is rested upon the apex of the predetermined spinous process 50, exactly in the point wherein it should penetrate the back of the patient.

The positioning of the sharp end 40 can be performed by availing of the control of a computerized monitoring system, for example of the O-ARM system, used in this 2D phase.

### Phase 2—Penetration (FIG. 3B)

The surgeon exerts pressing actions on the device 100 to implement the penetration of the sharp point 40 of the coupling means 2 in the predetermined spinous process 50, in particular by beating with a surgical hammer on the upper surface 31 of the head element 30.

### Phase 3—Tightening (FIGS. 3B and 3C)

When the sharp point 40 is inserted into the spinous process 50, one proceeds with screwing and tightening precisely the device 100 by means of wrench or another tightening tool. For example, a hexagon wrench can be used, referred to a head element 30 shaped like a hexagon nut.

The tightening is considered completed when the abutment surface 15 enters in contact with the skin 90 of the patient, or better it abuts thereon.

### Phase 4—Reference Assembly (FIG. 4)

Once completed the penetration and tightening phases, the position indicating means 60 is connected to the main body 10. With specific reference to the embodiment comprising the cap 6, the stem portion 8 is inserted into the hole 5, in centred position with respect to the head element 30 and the main body 10, and it is screwed up for the whole length of its thread within the hole 5.

Once ended the positioning of the cap 6, which acts as spatial reference or better as point of repère, one can proceed with the acquisition of the 3D images of the involved spinal district.

The present invention has been sofar described with reference to preferred embodiments. It is to be meant that other embodiments belonging to the same inventive core may exist, as defined by the protective scope of the herebelow reported claims.

1. A surgical device of repere configured to be connected to a vertebral spinous process of a patient,
said surgical device having a main body which comprises:
a skin contact element having a lower face and an upper face opposite to each other, wherein said lower face comprises an abutment surface shaped to abut on the skin of the patient at the vertebral spinous process;
a handling device connected to said skin contact element, configured to allow adjustment of the coupling between said surgical device and the vertebral spinous process, said handling device comprising a head element;
coupling element with the vertebral spinous process, connected to said skin contact element at said abutment surface and shaped to penetrate the external surface of the vertebral spinous process and insert at least partially inside thereof,
said coupling element comprising at least a protruding element with respect to said abutment surface;
wherein said surgical device further comprises position indicating elements configured to be connected to said main body,
the configuration of said surgical device being so that said position indicating elements, when said position indicating elements are connected to said main body, are arranged along a main development direction of said protruding element, the latter being an insertion preferential direction of said protruding element in the vertebral spinous process,
so that said position indicating elements indicate the penetration point of said protruding element in the vertebral spinous process.

2. The surgical device according to claim 1, wherein said skin contact element has a plate-shaped conformation.

3. The surgical device according to claim 1, wherein said abutment surface has a substantially flat or planar development.

4. The surgical device according to claim 1, wherein said head element is connected to said upper face in a permanent manner.

5. The surgical device according to claim 1, wherein said head element is shaped like a hexagonal nut.

6. The surgical device according to claim 1, wherein said head element comprises a concave external upper surface.

7. The surgical device according to claim 1, wherein said at least a protruding element has an overall oblong or stem-like conformation.

8. The surgical device according to claim 1, wherein said at least a protruding element extends according to a main development direction orthogonal with respect to said abutment surface.

9. The surgical device according to claim 1, wherein said at least a protruding element has a free terminal end having a sharp point.

10. The surgical device according to claim 1, wherein said at least a protruding element comprises a threaded external surface, being configured as a screw stem.

11. The surgical device according to claim 1, comprising a single protruding element having an axis of symmetry coinciding with said main development direction.

12. The surgical device according to claim 1, wherein said position indicating elements are arranged in centered position with respect to said main body.

13. The surgical device according to claim 1, having an axis of central symmetry which coincides with said main development direction and defines a preferential direction of insertion of said coupling elements in the vertebral spinous process.

14. The surgical device according to claim 1, wherein said position indicating elements comprise a plug element and said head element comprises at least one hole obtained at its own central portion and at said main development direction, said plug element being configured to engage reversibly with said at least one hole.
